(19) United States
(12) Patent Application Publication
(10) Pub. No.: US 2025/0329022 A1
(43) Pub. Date: Oct. 23, 2025

(54) PREOPERATIVE SUPPORT APPARATUS, PREOPERATIVE SUPPORT METHOD, AND NON-TRANSITORY COMPUTER-READABLE RECORDING MEDIUM STORING PREOPERATIVE SUPPORT PROGRAM

(71) Applicant: Konica Minolta, Inc., Tokyo (JP)

(72) Inventor: Sumiya NAGATSUKA, Tokyo (JP)

(21) Appl. No.: 19/182,806

(22) Filed: Apr. 18, 2025

(30) Foreign Application Priority Data
Apr. 22, 2024 (JP) 2024-069279

(51) Int. Cl. G06T 7/00; A61B 34/10; G06T 7/215; G16H 10/60; G16H 20/40; G16H 40/20

(52) U.S. Cl. CPC G06T 7/0016; A61B 34/10; G06T 7/215; G16H 10/60; G16H 20/40; G16H 40/20; A61B 2034/107; G06T 2207/10116; G06T 2207/30061; G06T 2207/30104

(57) ABSTRACT

Provided is a preoperative support apparatus that includes at least one hardware processor. The at least one hardware processor acquires a dynamic image analysis result of a patient and patient information on the patient and outputs preoperative support information for an operation based on the acquired dynamic image analysis result and the acquired patient information.

### CROSS REFERENCE TO RELATED APPLICATIONS

The present invention claims priority under 35 U.S.C. § 119 to Japanese Patent Application No. 2024-069279, filed on Apr. 22, 2024, the entire content of which is incorporated herein by reference.

### BACKGROUND

### Technological Field

The present invention relates to a preoperative support apparatus, a preoperative support method, and a non-transitory computer-readable recording medium storing a preoperative support program.

### Description of Related Art

In surgery, an examination or the like for acquiring a still image of a radiographic image is usually performed before the surgery. Further, an invention has also been proposed in which a dynamic image of a radiographic image is acquired before an operation and a surgical procedure of the operation is proposed based on the acquired dynamic image (for example, Japanese Patent Application Laid-Open No. 2021-115188).

A dynamic image has a larger amount of information than a still image. For this reason, it is considered that it is also possible to provide medical workers with various information and proposals based on information obtained from dynamic images. However, conventionally, utilization of information obtained from dynamic images has not been considered so much, and no information other than surgical procedures has been proposed for operations.

### SUMMARY

An object of the present invention is to provide a preoperative support apparatus, a preoperative support method, and a non-transitory computer-readable recording medium storing a preoperative support program, each of which is capable of providing information that allows improvement in efficiency, safety, and reliability of an operation to be attempted.

In order to achieve at least one of the above objects, a preoperative support apparatus reflecting one aspect of the present invention includes at least one hardware processor, and
the at least one hardware processor
   acquires a dynamic image analysis result of a patient and patient information on the patient, and
   outputs preoperative support information for an operation based on the acquired dynamic image analysis result and the acquired patient information.

In order to achieve at least one of the above objects, a preoperative support method reflecting one aspect of the present invention includes:
acquiring a dynamic image analysis result of a patient and patient information on the patient; and
outputting preoperative support information for an operation based on the acquired dynamic image analysis result and the acquired patient information.

In order to achieve at least one of the above objects, a non-transitory computer-readable recording medium storing a preoperative support program reflecting an aspect of the present invention is a non-transitory computer-readable recording medium storing a preoperative support program that causes a computer to execute:
acquiring a dynamic image analysis result of a patient and patient information on the patient; and
outputting preoperative support information for an operation based on the acquired dynamic image analysis result and the acquired patient information.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram describing an example of a configuration of a radiographic image processing system;

FIG. 2 is a block diagram illustrating an example of a functional configuration of an imaging controller in a radiographic image capturing apparatus constituting the radiographic image processing system;

FIG. 3 is a block diagram illustrating an example of a functional configuration of a radiography control apparatus constituting the radiographic image processing system;

FIG. 4 is a block diagram illustrating an example of a functional configuration of a radiographic image analyzing apparatus constituting the radiographic image processing system;

FIG. 5 is a block diagram illustrating an example of a functional configuration of a preoperative support apparatus according to the embodiment of the present invention;

FIG. 6 is a flowchart describing a preoperative support method performed by the preoperative support apparatus;

FIG. 7 is a diagram illustrating an exemplary dynamic image analysis result that the preoperative support apparatus acquires from the radiographic image analyzing apparatus;

FIG. 8 is a diagram illustrating another exemplary dynamic image analysis result that the preoperative support apparatus acquires from the radiographic image analyzing apparatus;

FIG. 9 is a diagram illustrating still another exemplary dynamic image analysis result that the preoperative support apparatus acquires from the radiographic image analyzing apparatus;

FIG. 10 is a diagram illustrating yet another exemplary dynamic image analysis result that the preoperative support apparatus acquires from the radiographic image analyzing apparatus;

FIG. 11 is a diagram illustrating an example of preoperative support information provided based on dynamic image analysis results and patient information;

FIG. 12 is a diagram schematically illustrating the bending and stretching of a knee joint; and

FIG. 13 is a diagram illustrating a knee artificial joint.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, an embodiment of the present invention will be described in detail with reference to the accompanying drawings.

### <Configuration of Radiographic Image Processing System>

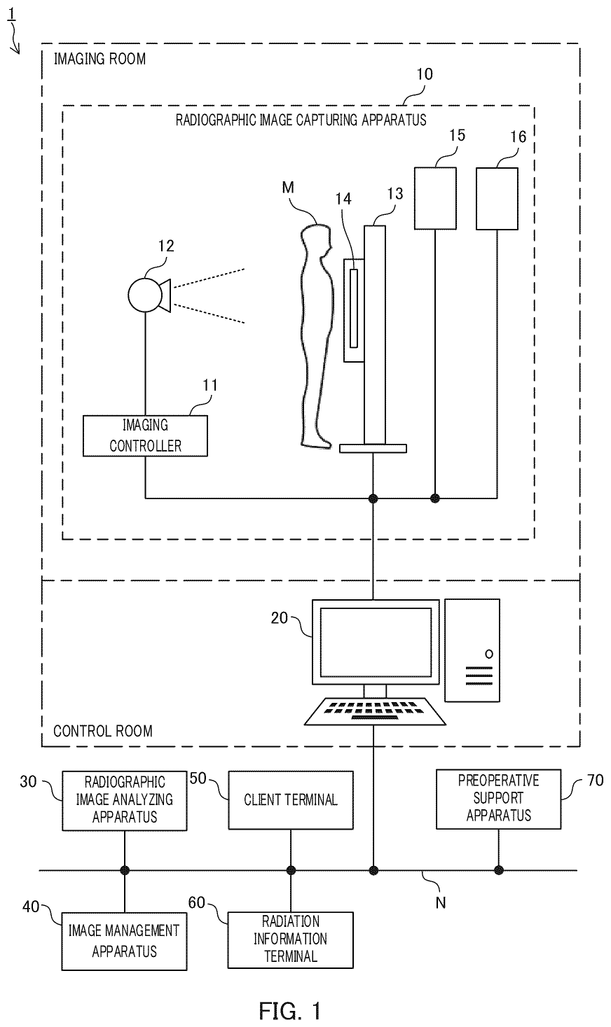

FIG. 1 is a diagram describing a radiographic image processing system 1 according to the present embodiment. The radiographic image processing system 1 includes a radiographic image capturing apparatus 10, a radiography control apparatus (console apparatus) 20, a radiographic image analyzing apparatus 30, an image management apparatus 40, and a client terminal 50.

In the example illustrated in FIG. 1, the radiographic image capturing apparatus 10 is disposed in an imaging room, and the radiography control apparatus 20 is disposed in a control room. The radiographic image capturing apparatus 10, the radiography control apparatus 20, the radiographic image analyzing apparatus 30, the image management apparatus 40, and the client terminal 50 are connected to each other via a communication network N. As the communication network N, for example, a communication network compliant with the Digital Image and Communications in Medicine (DICOM) standard or the like is used.

Further, a radiation information terminal 60 and a preoperative support apparatus 70 are connected to the communication network N. The radiation information terminal 60 is a radiation information system, for example, a radiology information system (RIS), which transmits information on a radiation examination, for example, examination order information of a patient or the like, to the radiographic image processing system 1. The preoperative support apparatus 70 provides preoperative support information based on an analysis result of a dynamic image (hereinafter referred to as “dynamic image analysis result”) and patient information which are inputted from the radiographic image processing system 1.

The radiographic image capturing apparatus 10 captures a dynamic image, which is a radiographic image, based on the control of the radiography control apparatus 20. The radiography control apparatus 20 controls the radiographic image capturing apparatus 10 based on examination order information or the like including patient information or the like transmitted from the radiation information terminal 60. A dynamic image generated by the radiographic image capturing apparatus 10 is subjected to predetermined processing by the radiography control apparatus 20 and is transmitted to the radiographic image analyzing apparatus 30. The radiographic image analyzing apparatus 30 executes a dynamic analysis on a dynamic image. A dynamic image and a dynamic image analysis result are transmitted to and managed by the image management apparatus 40 as a medical image management system, for example, a picture archiving and communication system (PACS) or the like.

A dynamic image, a dynamic image analysis result, and the like are transmitted to the client terminal 50 and viewed by a medical worker such as a doctor. In addition, a dynamic image, a dynamic image analysis result, patient information, and the like are transmitted to the preoperative support apparatus 70, and preoperative support information is provided to a medical worker such as a doctor.

Each of the radiographic image capturing apparatus 10, the radiography control apparatus 20, and the radiographic image analyzing apparatus 30 is a kind of computer that includes a processor and a memory and implements a predetermined function by reading, developing, and executing a program stored in the memory.

### [Radiographic Image Capturing Apparatus 10]

As illustrated in FIG. 1, the radiographic image capturing apparatus 10 includes an imaging controller 11, a radiation irradiator 12, an imaging table 13, a radiation detector 14, a display 15, and a sound outputter 16.

The imaging controller 11 acquires setting information on settings of radiation dynamic imaging (hereinafter referred to as “dynamic imaging”) from the radiography control apparatus 20. The imaging controller 11 sets imaging conditions for performing dynamic imaging based on the setting information and controls the radiation irradiator 12 based on the imaging conditions to cause a patient M (subject) to be irradiated with radiation and imaging to be performed. The imaging controller 11 is constituted by a central processing unit (CPU), a random access memory (RAM), a read only memory (ROM), and the like.

The setting information is information on settings for performing dynamic imaging on the patient M. The setting information includes, for example, at least one of a plurality of types of dynamic analyses that can be executed on a dynamic image by the radiographic image analyzing apparatus 30. In a case where a plurality of types of dynamic analyses is combined, the setting information may also include information on the combination. The setting information is set by the operating person of the radiographic image processing system 1, for example, an imaging technician or the like, in the radiography control apparatus 20 to be described later.

The imaging conditions include, for example, various conditions such as a pulse rate, a pulse width, a pulse interval, the number of imaging frames per imaging, a dose per unit time of radiation irradiation, and a body state (a respiratory state or the like) of the patient M. The pulse rate is the number of times of radiation irradiation per second and coincides with the frame rate of image data. The pulse width is a radiation irradiation time per radiation irradiation. The pulse interval is a time from the start of one radiation irradiation to the start of the next radiation irradiation and coincides with a time interval (frame interval) between a plurality of image data. The imaging conditions may be automatically determined by the imaging controller 11 of the radiographic image capturing apparatus 10 based on the setting information.

The radiation irradiator 12 is disposed at a position facing the radiation detector 14 fixed to the imaging table 13. The radiation irradiator 12 irradiates radiation (X-rays) according to the control of the imaging controller 11.

The radiation detector 14 is constituted by a semiconductor image sensor, such as a flat panel detector (FPD). The radiation detector 14 includes a board in which a plurality of detection elements (pixels) that detects the radiation irradiated by the radiation irradiator 12 according to the intensity of the radiation, converts the detected radiation into an electric signal, and accumulates the electric signal is arranged in a matrix. Each pixel of the board is constituted by, for example, a switcher such as a thin film transistor (TFT).

The radiation detector 14 controls the switcher of each pixel based on image reading conditions inputted through the radiography control apparatus 20 to read electric signals accumulated in each pixel, and outputs intensity information for each pixel to an image generator 113. The image reading conditions are, for example, a frame rate, a frame interval, a pixel size, an image size (matrix size), and the like. The frame rate is the number of frame images acquired per second and coincides with the pulse rate. The frame interval is a time from the start of one operation of acquiring image data to the start of the operation of acquiring the next frame image and coincides with the pulse interval.

The imaging controller 11 and the radiation detector 14 are connected to each other, and are configured to exchange synchronization signals with each other to synchronize the radiation irradiation operation and the image reading operation.

As described above, the radiographic image capturing apparatus 10 performs dynamic imaging of a radiographic image by the radiation irradiator 12 irradiating radiation and the radiation detector 14 generating image data based on the intensity of the irradiated radiation under the control of the imaging controller 11.

The display 15 and the sound outputter 16 provide the patient M with instructions on a posture to be taken, a body state, a respiratory state, and the like when dynamic imaging of the patient M is performed. The display 15 is, for example, a display apparatus such as a cathode ray tube (CRT), a liquid crystal display, or an organic electro luminescence (EL) display. The sound outputter 16 is, for example, a sound output apparatus such as a speaker. Each of the display 15 and the sound outputter 16 may provide instructions having the same content to the patient M, or only one of the display 15 and the sound outputter 16 may provide an instruction to the patient M.

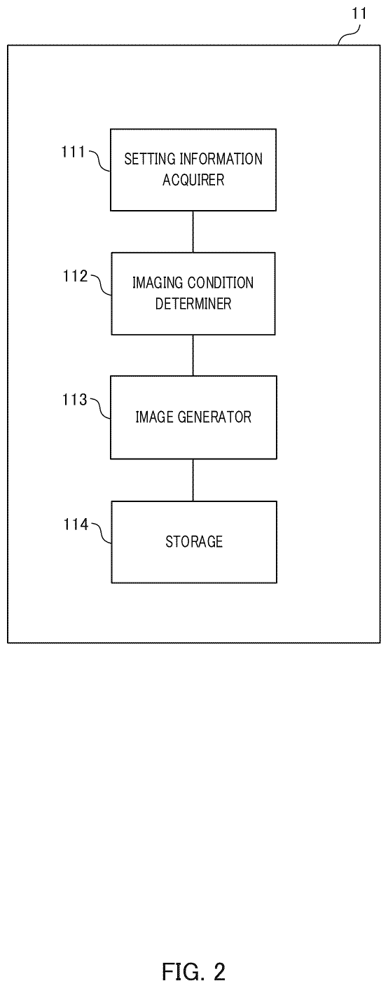

FIG. 2 is a block diagram illustrating an example of a functional configuration of the imaging controller 11 in the radiographic image capturing apparatus 10 constituting the radiographic image processing system 1. The imaging controller 11 includes a setting information acquirer 111, an imaging condition determiner 112, the image generator 113, and a storage 114.

The setting information acquirer 111 acquires setting information from the radiography control apparatus 20.

The imaging condition determiner 112 determines, based on the setting information, imaging conditions when dynamic imaging of the patient M is performed. Information indicating a correspondence relationship between a plurality of types of dynamic analyses and imaging conditions suitable for the respective dynamic analyses is stored in the storage 114 in advance. Further, information indicating a correspondence relationship between a combination of a plurality of types of dynamic analyses and imaging conditions suitable for the combination is also stored in the storage 114 in advance. The imaging condition determiner 112 may determine the imaging conditions by reading information, which indicates the correspondence relationship for a dynamic analysis or a combination of a plurality of types of dynamic analyses indicated by the setting information, from the storage 114 and collating the information with the setting information.

Note that, for example, in the case of screening, emergency, or the like, a dynamic analysis serving as the setting information may not be set. In such a case, the imaging condition determiner 112 determines the imaging condition(s) by causing the operating person to select at least one imaging condition from a plurality of predefined imaging conditions. The imaging condition determiner 112 also allows the operating person to select examination order information and determines the imaging condition(s) based on the selected examination order information. As described above, in a case where a dynamic analysis cannot be set before dynamic imaging, a dynamic analysis is set after dynamic imaging under the imaging condition(s) selected by the operating person, and the dynamic analysis is executed in the radiographic image analyzing apparatus 30 to be described later.

The image generator 113 executes dynamic imaging on the patient M based on the determined imaging condition(s) and generates a radiographic image with a plurality of frames. Specifically, the image generator 113 controls the operations of the radiation irradiator 12 and the radiation detector 14 based on the imaging condition(s), and generates image data by acquiring, for each pixel, intensity information on the intensity of radiation transmitted through the subject from the radiation detector 14.

As described above, the storage 114 stores, in advance, information indicating a correspondence relationship between a plurality of types of dynamic analyses and imaging conditions suitable for the respective dynamic analyses, information indicating a correspondence relationship between a combination of a plurality of types of dynamic analyses and imaging conditions suitable for the combination, and the like.

### [Radiography Control Apparatus 20]

The radiography control apparatus 20 is, for example, a computer such as a personal computer (PC) or a workstation. The radiography control apparatus 20 may be a desktop computer as in the example illustrated in FIG. 1 or may be a portable computer such as a laptop computer or a tablet computer.

The radiography control apparatus 20 receives examination order information from the radiation information terminal 60 or the like and transmits the examination order information to the radiographic image capturing apparatus 10 to control the dynamic imaging of the radiographic image capturing apparatus 10.

The examination order information includes various information on dynamic imaging to be executed next, such as instruction information on respiration, patient information, examination information, imaging information, and data attributes. The examination information includes information on an examination ID, an examination target site (for example, the chest, in particular the lungs, the heart or the like), and the type of analysis (for example, ventilation analysis, pulmonary blood flow analysis, measurement of the maximum ventilation volume, and the like). The examination order information is generated, for example, when a doctor or the like requests the radiographic image processing system 1 to perform dynamic imaging of the patient M, or the like.

In addition, the radiography control apparatus 20 generates setting information indicating at least one dynamic analysis among a plurality of types of dynamic analyses executable by the radiographic image analyzing apparatus 30 based on an input of the operating person. In the case of combining a plurality of types of dynamic analyses, the radiography control apparatus 20 generates setting information indicating the combination of the plurality of types of dynamic analyses. The operating person recognizes which dynamic analyses are to be combined among the plurality of types of dynamic analyses by referring to, for example, the content of the examination order information and performs an input operation for generating the setting information based on the recognition. Alternatively, the operating person may recognize which dynamic analyses are to be combined, based on information transmitted from a doctor or the like by another method.

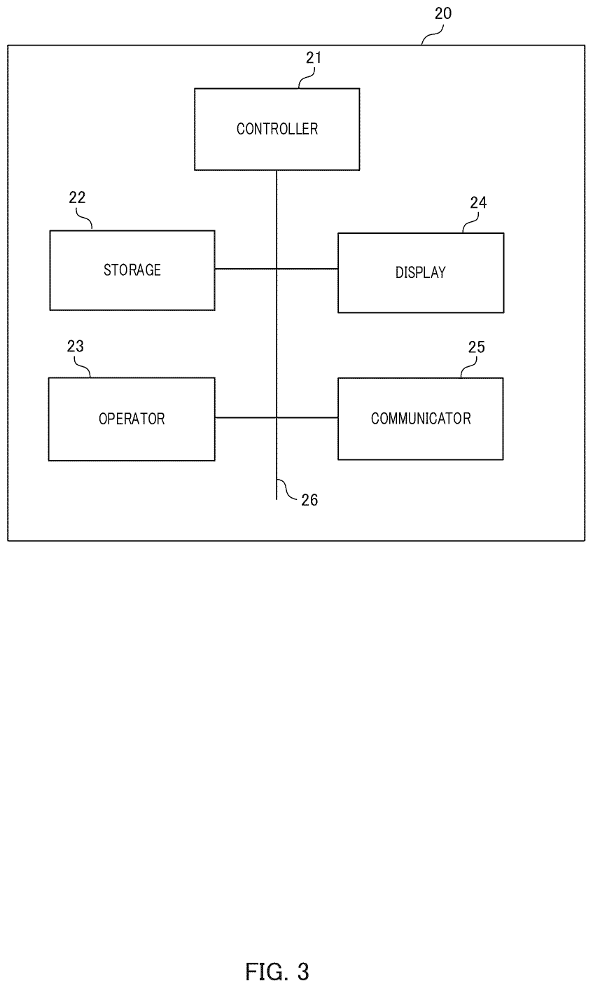

FIG. 3 is a block diagram illustrating an example of a functional configuration of the radiography control apparatus 20 constituting the radiographic image processing system 1. The radiography control apparatus 20 includes a controller 21, a storage 22, an operator 23, a display 24, and a communicator 25. The respective configurations included in the radiography control apparatus 20 are connected to each other by a bus 26.

The radiography control apparatus 20 outputs, to the radiographic image capturing apparatus 10, setting conditions set by the operating person or the like and examination order information acquired in advance from the radiation information terminal 60 or the like, and controls imaging processing by the radiographic image capturing apparatus 10. The radiography control apparatus 20 may display a dynamic image generated by the radiographic image capturing apparatus 10, for example, for the operating person to confirm.

The controller 21 is constituted by a CPU, a RAM, and the like. In the controller 21, in response to an operation of the operator 23, the CPU reads a system program and various processing programs stored in the storage 22, develops the system program and various processing programs in the RAM, and controls the operation of each unit of the radiography control apparatus 20 based on the developed programs.

The storage 22 is constituted by a non-volatile semiconductor memory, a hard disk, or the like. The storage 22 stores various programs to be executed by the controller 21, parameters necessary for the execution of processing by the programs, or data such as processing results. The various programs are stored in the form of readable program codes, and the controller 21 sequentially executes operations according to the program codes.

Further, the storage 22 stores image reading conditions for performing dynamic imaging. Further, the storage 22 stores examination order information transmitted from the radiation information terminal 60 or the like. When the radiography control apparatus 20 controls the dynamic imaging of the radiographic image capturing apparatus 10, the radiography control apparatus 20 reads the image reading conditions and examination order information corresponding to the patient M from the storage 22 and transmits the read image reading condition and the read examination order information.

The operator 23 is an operation device such as a keyboard including a cursor key, number input keys, various function keys and the like, a pointing device such as a mouse or a trackball, and a touch screen. The operator 23 generates an instruction signal based on an input of the operating person, and outputs the instruction signal to the controller 21.

The display 24 is constituted by a display device such as a CRT, a liquid crystal display, or an organic EL display. The display 24 displays an input instruction from the operator 23, image data generated by the radiographic image capturing apparatus 10, and the like according to an instruction of a display signal inputted from the controller 21.

The communicator 25 transmits and receives data to and from the radiographic image capturing apparatus 10, the radiographic image analyzing apparatus 30, the radiation information terminal 60, and the like.

### [Radiographic Image Analyzing Apparatus 30]

The radiographic image analyzing apparatus 30 is, for example, a computer such as a PC or a workstation. The radiographic image analyzing apparatus 30 may be a desktop computer or a portable computer, such as a laptop computer or a tablet computer.

The radiographic image analyzing apparatus 30 executes a dynamic analysis on a dynamic image captured by the radiographic image capturing apparatus 10 based on the setting information set in the radiography control apparatus 20. The radiological image analyzing apparatus 30 may execute a plurality of types of dynamic analyses in combination.

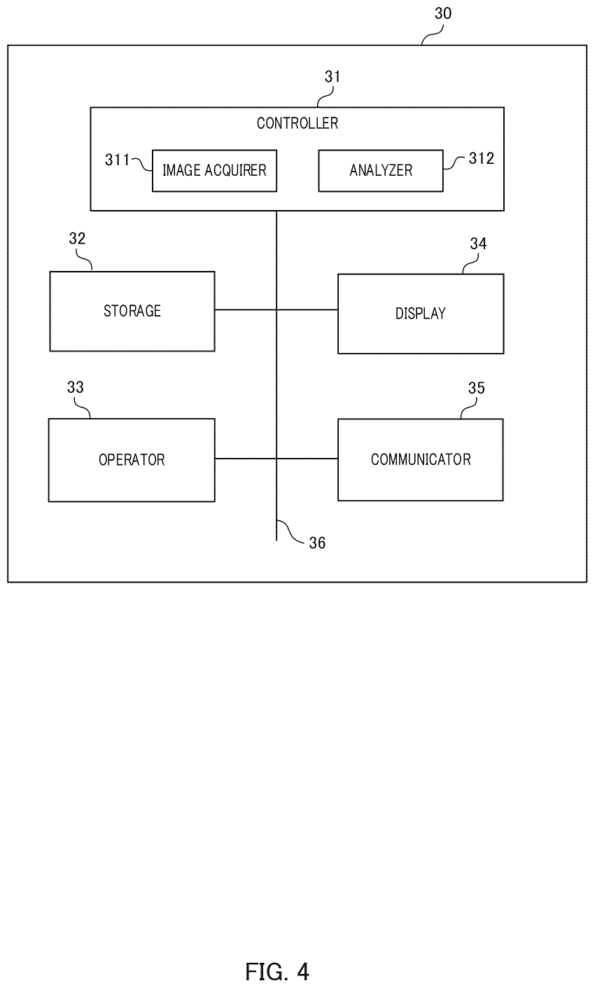

FIG. 4 is a block diagram illustrating an example of a functional configuration of the radiographic image analyzing apparatus 30 constituting the radiographic image processing system 1. The radiographic image analyzing apparatus 30 includes a controller 31, a storage 32, an operator 33, a display 34, and a communicator 35. The respective configurations included in the radiographic image analyzing apparatus 30 are connected to each other by a bus 36.

The controller 31 is constituted by a CPU, a RAM, and the like. In the controller 31, in response to an operation of the operator 33, the CPU reads a system program and various processing programs stored in the storage 32, develops the system program and various processing programs in the RAM, and executes operation control of each unit of the radiographic image analyzing apparatus 30, a dynamic analysis, and the like based on the developed programs.

The controller 31 includes an image acquirer 311 and an analyzer 312.

The image acquirer 311 acquires a dynamic image which is a radiographic image with a plurality of frames generated by the radiographic image capturing apparatus 10.

The analyzer 312 executes the dynamic analysis set in the setting information, on the dynamic image acquired from the radiographic image capturing apparatus 10, and acquires an analysis result. At this time, in a case where the analyzer 312 cannot analyze the dynamic image (cannot acquire an analysis result), the analyzer 312 determines that the analysis is impossible. The analysis by the analyzer 312 may be performed with a predetermined algorithm or using artificial intelligence (AI).

The analyzer 312 has, for example, a blood flow analysis mode, a ventilation analysis mode, an adhesion analysis mode, a diaphragm movement amount analysis mode, an orthopedic-related measurement mode, and the like as the types of dynamic analyses. Each mode will be briefly described below.

The blood flow analysis mode is a mode in which a signal change in the lung field synchronized with the heartbeat is visualized.

The ventilation analysis mode is a mode in which a signal change in a time direction in a specific time-frequency band is extracted and a lung tissue behavior during respiration is visualized.

The adhesion analysis mode is a mode in which the degree of adhesion of tissues is visualized.

The diaphragm movement amount analysis mode is a mode in which the upward/downward movement of the diaphragm associated with respiration is tracked.

The orthopedic-related measurement mode is, for example, a mode in which a change in the position of a specified bone in the four limbs or the like is measured and the trajectory of the movement is displayed.

The storage 32 is constituted by a non-volatile semiconductor memory, a hard disk, or the like. The storage 32 stores various programs to be executed by the controller 31, parameters necessary for the execution of processing by the programs, or data such as processing results. The various programs are stored in the form of readable program codes, and the controller 31 sequentially executes operations according to the program codes.

The storage 32 also stores patient information, examination information, and list information indicating the status (for example, the progress status such as during reception, during dynamic analysis, and completion of analysis) according to each dynamic image generated by the radiographic image capturing apparatus 10. Further, analysis results are stored in the storage 32 in association with the dynamic images.

The operator 33 is an operation device such as a keyboard including a cursor key, number input keys, various function keys and the like, a pointing device such as a mouse or a trackball, and a touch screen. The operator 33 generates an instruction signal based on an input by the operating person, and outputs the instruction signal to the controller 31. Further, the operator 33 may include a touch screen on the display screen of the display 34, and in this case, outputs an instruction signal inputted through the touch screen to the controller 31.

The display 34 is constituted by a display device such as a CRT, a liquid crystal display, or an organic EL display. The display 34 displays an input instruction from the operator 33, image data generated by the radiographic image capturing apparatus 10, and the like according to an instruction of a display signal inputted from the controller 31.

The communicator 35 transmits and receives data to and from the radiography control apparatus 20, the image management apparatus 40, the preoperative support apparatus 70, and the like.

### [Preoperative Support Apparatus 70]

The preoperative support apparatus 70 is, for example, a computer such as a PC or a workstation. The preoperative support apparatus 70 may be a desktop computer or a portable computer, for example, a laptop computer or a tablet computer.

The preoperative support apparatus 70 provides preoperative support information based on a dynamic image analysis result and patient information which are inputted from the radiographic image processing system 1.

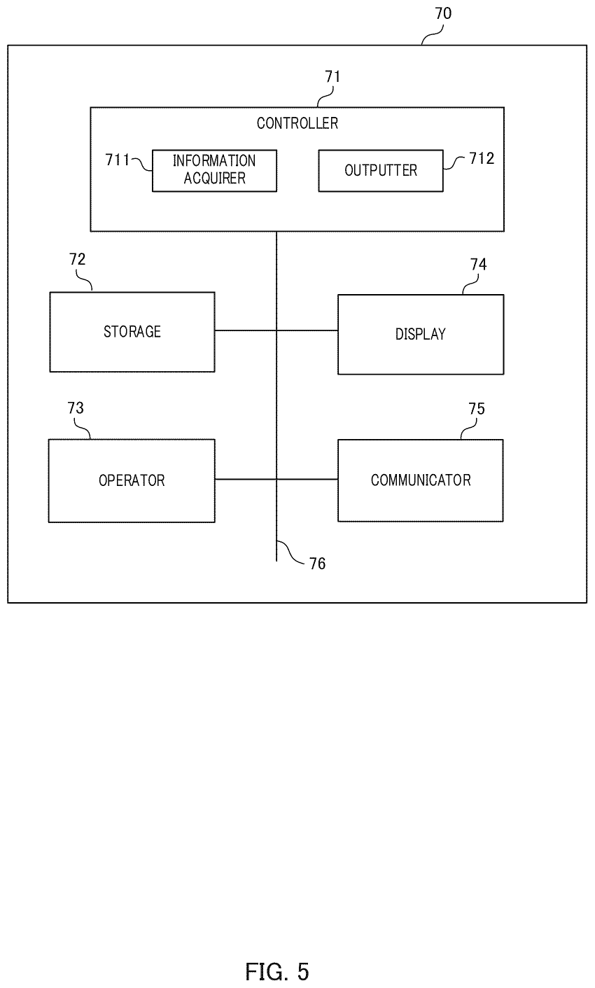

FIG. 5 is a block diagram illustrating an example of a functional configuration of the preoperative support apparatus 70. The preoperative support apparatus 70 includes a controller 71, a storage 72, an operator 73, a display 74, and a communicator 75. The respective configurations included in the preoperative support apparatus 70 are connected by a bus 76.

The controller 71 is, for example, a computer including at least one hardware processor and is constituted by a CPU, a RAM, and the like. The preoperative support program is stored in a non-transitory computer-readable recording medium, and the storage 72 stores the preoperative support program from the recording medium. In the controller 71, in response to an operation of the operator 73, the CPU reads a system program and various processing programs stored in the storage 72, develops the system program and various processing programs in the RAM, and executes processing for providing preoperative support information based on the developed programs.

The controller 71 includes an information acquirer 711 and an outputter 712. In the controller 71, at least one hardware processor functions as the information acquirer 711 and the outputter 712.

The information acquirer 711 acquires, for the patient to be examined, a dynamic image analysis result analyzed by the analyzer 312 of the radiographic image analyzing apparatus 30 and patient information included in examination order information inputted through the radiation information terminal 60.

In the present embodiment, a dynamic image analysis result includes change information on a change in an interest region in a dynamic image. The change information is, for example, a movement amount of an interest region in a dynamic image, and in a case where the dynamic image is a dynamic image of the chest, the change information is an area of a region in which a movement amount of the lung field associated with respiration is smaller than a threshold, a change amount related to ventilation of the lung field associated with respiration or a blood flow of the lung field associated with respiration, or the like.

The outputter 712 acquires preoperative support information for an operation based on the acquired dynamic image analysis result and patient information, and outputs the preoperative support information to the display 74 to be described later. For example, the outputter 712 determines the state of an interest region based on the change information of the interest region and the patient information as described above and acquires the preoperative support information for the interest region. The outputter 712 may acquire the preoperative support information with a predetermined algorithm or by using AI.

The storage 72 is constituted by a non-volatile semiconductor memory, a hard disk, or the like. The storage 72 stores various programs to be executed by the controller 71, parameters necessary for the execution of processing by the programs, or data such as processing results. The various programs are stored in the form of readable program codes, and the controller 71 sequentially executes operations according to the program codes.

Further, the storage 72 stores reference information on preoperative support information. The reference information includes a surgical procedure of an operation, a tool to be used in the operation, an operative time required for the operation, an operating room, the required number of surgical staff, and the like, and is stored in the storage 72, for example, based on past operation cases. The outputter 712 acquires appropriate preoperative support information by referring to the reference information stored in the storage 72 together with the acquired dynamic image analysis result and patient information, and outputs the acquired preoperative support information.

The operator 73 is an operation device such as a keyboard including a cursor key, number input keys, various function keys and the like, a pointing device such as a mouse or a trackball, and a touch screen. The operator 73 generates an instruction signal based on an input of the operating person, and outputs the instruction signal to the controller 71. In addition, the operator 73 may include a touch screen on the display screen of the display 74, and in this case, outputs an instruction signal inputted through the touch screen to the controller 71.

The display 74 is constituted by a display device such as a CRT, a liquid crystal display, or an organic EL display. The display 74 displays an input instruction from the operator 73, preoperative support information outputted by the outputter 712, and the like according to an instruction of a display signal inputted from the controller 71.

The communicator 75 transmits and receives data to and from the radiographic image analyzing apparatus 30 or the like.

### [Preoperative Support Method]

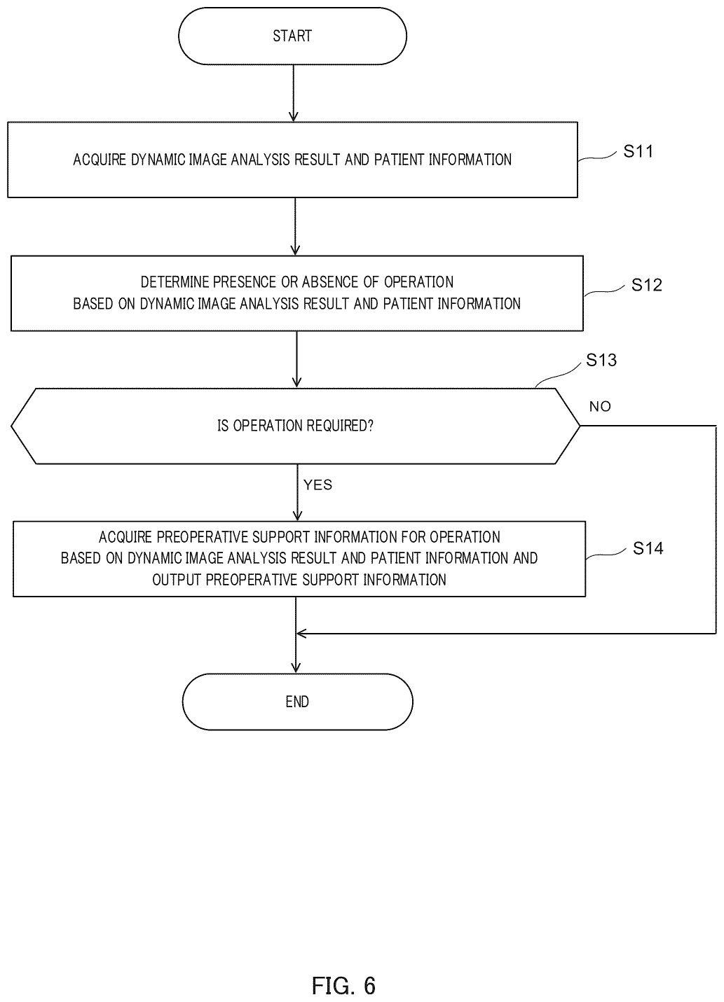

FIG. 6 is a flowchart describing a preoperative support method performed by the preoperative support apparatus 70. The preoperative support method is implemented by causing the preoperative support apparatus 70 to execute a preoperative support program.

Note that, before the processing illustrated in FIG. 6, the input of the examination order information from the radiation information terminal 60, the dynamic imaging of the patient M by the radiographic image capturing apparatus 10 and the radiography control apparatus 20, and the analysis of a dynamic image by the radiographic image analyzing apparatus 30 have been completed.

### (Step S11)

The controller 71 (information acquirer 711) acquires a dynamic image analysis result from the radiographic image analyzing apparatus 30 and acquires examination order information including patient information and the like from the radiation information terminal 60.

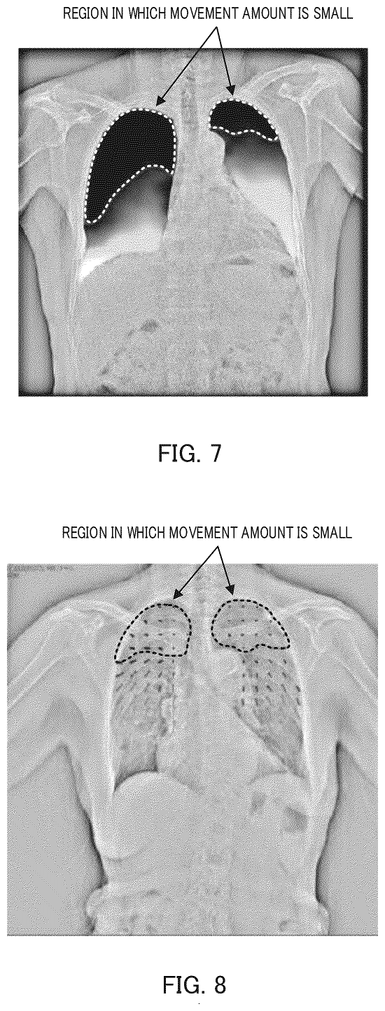

The controller 71 (information acquirer 711) acquires, for example, a dynamic image analysis result as exemplified in FIG. 7 to FIG. 10 to be described later from the radiographic image analyzing apparatus 30. Further, the controller 71 (information acquirer 711) acquires the patient information included in the examination order information from the radiographic image analyzing apparatus 30. The patient information includes, for example, as exemplified in FIG. 10 to be described later, the age, sex, chronic diseases, complications, contraindications, medication content, and the like of the patient M.

### (Step S12)

The controller 71 (outputter 712) determines the presence or absence of an operation on the patient M based on the dynamic image analysis result and the patient information.

Here, FIG. 7 to FIG. 10 are diagrams illustrating some exemplary dynamic image analysis results that the preoperative support apparatus 70 acquires from the radiographic image analyzing apparatus 30.

FIG. 7 and FIG. 8 illustrate the results of analyzing dynamic images by using the adhesion analysis mode as the analysis mode of dynamic images. In FIG. 7, signal value patterns in the lung field are extracted by the analysis of the dynamic image, and the movement amount of each region associated with respiration is measured, whereby the tendency of the movement of each region is displayed in a plurality of colors but is displayed in grayscale for convenience of the drawing. Further, in FIG. 8, signal value patterns in the lung field are extracted by the analysis of the dynamic image, and the movement amount of each region associated with respiration is displayed by vectors in a plurality of colors by using the maximum inspiratory level frame as a reference frame but is displayed in grayscale for convenience of the drawing.

In FIG. 7, for easier understanding, a region in which the movement amount is smaller than a predetermined movement threshold is surrounded by a white dot line. The controller 71 (outputter 712) determines the presence or absence of an operation based on the smallness of a movement amount, the area of a region in which a movement amount is small, or the like. Further, even in FIG. 8, for easier understanding, a region in which a vector is smaller than a predetermined size is surrounded by a black dotted line. The controller 71 (outputter 712) determines the presence or absence of an operation based on the smallness of a movement amount, the area of a region in which a movement amount is small, or the like.

The controller 71 (outputter 712) can determine the degree of adhesion or invasion of the lung field tissue based on the dynamic image analysis results illustrated in FIG. 7 and FIG. 8, and determines the presence or absence of an operation based on the degree of adhesion or invasion of the lung field tissue. At this time, the controller 71 (outputter 712) also refers to the patient information to determine the presence or absence of an operation.

As for the adhesion of the lung field tissue, the degree of adhesion can be determined by the ratio between the lung field area at the time of maximum inspiration and the area of a region in which a movement amount is small. In addition, as for the invasion of the lung field tissue, the invasion portion of the lung field tissue and the ribs move in synchronization with each other, associated with respiration, and thus, when the dynamic image is analyzed for the synchronization with the movement of the ribs, the degree of invasion can be determined, and the adhesion and invasion of the lung field tissue can be distinguished.

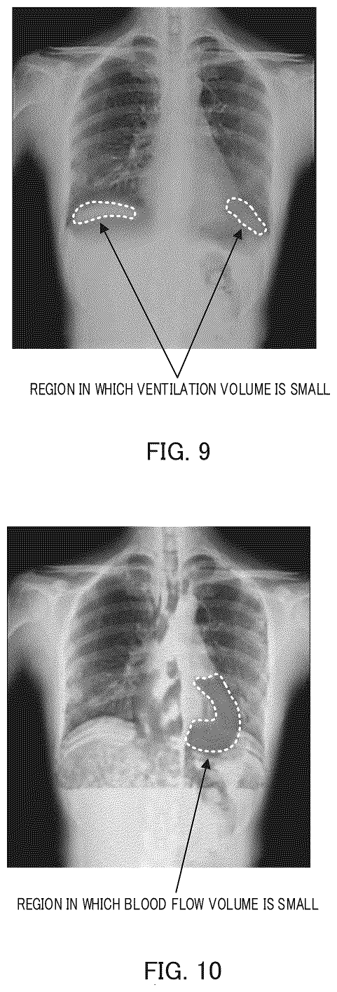

Further, FIG. 9 illustrates the result of analyzing a dynamic image by using the ventilation analysis mode as the analysis mode of the dynamic image. In FIG. 9, signal value changes in the lung tissue associated with respiration are extracted by the analysis of the dynamic image, and the signal value changes by using the maximum inspiratory level frame as a reference frame are displayed with the density of a single color, but are displayed in grayscale for convenience of the drawing.

Even in FIG. 9, for easier understanding, a region in which a signal value change is smaller than a predetermined change threshold, that is, a region in which a ventilation volume is small is surrounded by a white dot line. The controller 71 (outputter 712) determines the presence or absence of an operation based on the smallness of a signal value change, the area of a region in which a signal value change is small, or the like.

The controller 71 (outputter 712) can determine the degree of a chronic respiratory disease based on the dynamic image analysis result illustrated in FIG. 9, and determines the presence or absence of an operation based on the degree of the chronic respiratory disease. At this time, the controller 71 (outputter 712) also refers to the patient information to determine the presence or absence of an operation. The chronic respiratory disease is, for example, a chronic obstructive pulmonary disease (COPD), interstitial pneumonia, or the like.

Further, FIG. 10 illustrates the result of analyzing a dynamic image by using the blood flow analysis mode as the analysis mode of the dynamic image. In FIG. 10, a signal value change in the lung tissue synchronized with the heartbeat is extracted by the analysis of the dynamic image and is displayed with the density of a single color according to the similarity of the signal value change with respect to the heartbeat, but is displayed in grayscale for convenience of the drawing.

Even in FIG. 10, for easier understanding, a region in which the similarity of a signal value change with respect to the heartbeat is smaller than a predetermined similarity threshold, that is, a region in which a blood flow volume is small is surrounded by a white dot line. The controller 71 (outputter 712) determines the presence or absence of an operation based on the smallness of the similarity, the area of a region in which the similarity is small, or the like.

The controller 71 (outputter 712) can determine the degree of pulmonary embolism or pulmonary hypertension based on the dynamic image analysis result illustrated in FIG. 10, and determines the presence or absence of an operation based on the degree of the pulmonary embolism or pulmonary hypertension. At this time, the controller 71 (outputter 712) also refers to the patient information to determine the presence or absence of an operation.

In addition, when the controller 71 (outputter 712) determines the presence or absence of an operation, the controller 71 (outputter 712) may determine the presence or absence of an operation based on not only one analysis result but also a plurality of analysis results. For example, the presence or absence of an operation may be determined based on different analysis results by using the same analysis mode, or the presence or absence of an operation may be determined based on different analysis results by using different analysis modes. By determining the presence or absence of an operation based on a plurality of analysis results, the controller 71 (outputter 712) can perform more accurate determination.

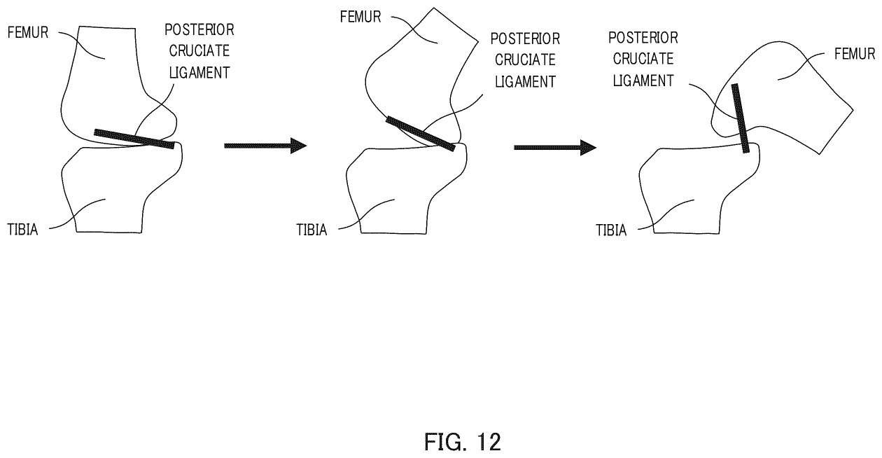

In addition, in FIG. 7 to FIG. 10, the chest (lungs) is the examination target, but for example, a joint may be set as the examination target, a dynamic image may be captured, and the presence or absence of an operation on the patient M may be determined based on a dynamic image analysis result and patient information. For example, in a case where a knee joint is the examination target, dynamic imaging is performed while bending and stretching the knee joint (see FIG. 12 to be described later). Then, the controller 71 (outputter 712) may determine the presence or absence of an operation by analyzing a dynamic image, measuring, for example, a positional relationship between the femur and the tibia as a dynamic image analysis result, and determining whether the function of the knee joint is normal based on the measurement result.

### (Step S13)

In a case where an operation is required (YES), the controller 71 (outputter 712) proceeds to step S14, and in a case where an operation is not required (NO), the controller 71 ends the series of processing.

### (Step S14)

The controller 71 (outputter 712) refers to reference information stored in the storage 72 together with the dynamic image analysis result and the patient information, acquires preoperative support information necessary for an operation, and outputs the preoperative support information to the display 74.

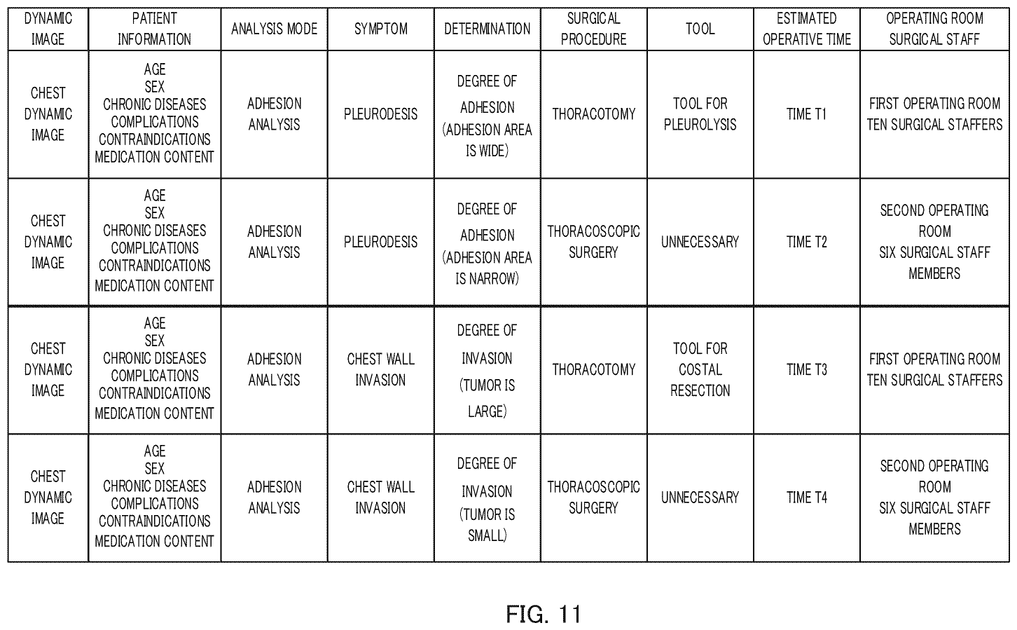

Here, preoperative support information will be described with reference to FIG. 11. FIG. 11 is a diagram illustrating an example of preoperative support information provided based on dynamic image analysis results and patient information. Note that, here, an example in which a dynamic image of the chest is captured and the dynamic image is analyzed in the adhesion analysis mode will be described.

In a case where a dynamic image of the chest is captured and the dynamic image is analyzed in the adhesion analysis mode, the dynamic image analysis results illustrated in FIG. 7 and FIG. 8 can be obtained. Although the controller 71 (outputter 12) determines the presence or absence of an operation by using the dynamic image analysis result in step S12, the controller 71 (outputter 12) acquires the preoperative support information by using the same dynamic image analysis result in step S14.

Specifically, the controller 71 (outputter 12) refers to reference information stored in the storage 72 based on the degree of adhesion or invasion of the lung field tissue determined in step S12, and acquires preoperative support information necessary for an operation, for example, a surgical procedure, a tool, and the like of the operation.

For example, in a case where a lung reoperation or a lung operation on a patient having a case with a history of pneumonia or the like is performed, it is necessary to select a surgical procedure or a tool for the operation according to the degree of pleurodesis. For example, in a case where the adhesion range of pleurodesis is wide, a thoracotomy is selected. In this case, a tool for pleurolysis is required at a site where the adhesion exists.

On the other hand, in a case where the adhesion range of pleurodesis is narrow, thoracoscopic surgery is selected. In this case, since the site where the pleurodesis is present can be known from a dynamic image analysis result, an endoscope can be inserted while avoiding the site. This is also useful when an endoscopic surgery support robot is used in a case where thoracoscopic surgery is performed. For example, a dynamic image analysis result includes positional information on the position of an interest region, and the controller 71 (outputter 712) outputs the positional information as preoperative support information.

Further, for example, in a case where an operation for lung cancer is performed, it is necessary to select a surgical procedure and a tool of the operation according to the degree of chest wall invasion. For example, in a case where the degree of chest wall invasion is large, that is, in a case where the tumor is large, a thoracotomy is selected. In this case, since costal combined resection is required at the site invaded by the tumor, a tool for performing costal combined resection is required. Further, since a cardiovascular surgeon is required as surgical staff in a case where there is aortic invasion, the need for a cardiovascular surgeon is provided as preoperative support information.

On the other hand, in a case where the degree of chest wall invasion is small or there is no chest wall invasion, that is, in a case where the tumor is small, thoracoscopic surgery is selected. In this case, since the site where the tumor is present is known from a dynamic image analysis result, an endoscope can be inserted from an appropriate position. This is also useful when an endoscopic surgery support robot is used in a case where thoracoscopic surgery is performed. For example, a dynamic image analysis result includes positional information on the position of an interest region, and the controller 71 (outputter 712) outputs the positional information as preoperative support information.

As described above, when the surgical procedure and the tools to be used of the surgery can be selected, the controller 71 (outputter 712) can determine the estimated operative time, the operating room to be used, and the required number of surgical staff by referring to the reference information stored in the storage 72, for example.

In FIG. 11, an example in which a dynamic image of the chest is captured and preoperative support information is acquired based on a dynamic image analysis result obtained by analyzing the dynamic image in the adhesion analysis mode has been described, but preoperative support information can be similarly acquired for a joint. Here, as an example, a case where a knee joint is the examination target will be described. FIG. 12 is a diagram schematically illustrating the bending and stretching of a knee joint.

In a case where a knee joint is the examination target, dynamic imaging is performed while bending and stretching the knee joint. The controller 71 (outputter 712) analyzes dynamic images, measures the degree of deformation of the knee as a dynamic image analysis result, and acquires, based on the degree of deformation of the knee, preoperative support information necessary for an operation, for example, a surgical procedure, a tool, and the like of the operation.

For example, in a case where the degree of deformation of the knee is small, arthroscopic surgery is selected. In this case, in the knee joint, it is necessary to remove a damaged meniscus or cartilage, repair a damaged cartilage or a torn meniscus, or the like, and therefore, tools to be used in an operation for the above are required. Further, since a dynamic image analysis result includes positional information on the position of an interest region, the controller 71 (outputter 712) outputs the positional information of the surgical site as preoperative support information.

In a case where the degree of deformation of the knee is large to some extent, osteotomy is selected. In this case, in order to change the positional relationship between the femur and the tibia, it is necessary to incise the front surface of the knee, cut the tibia in the vicinity of the knee joint, or the like, and thus, tools to be used in an operation for the above are required.

In a case where the degree of deformation of the knee is much larger, total knee replacement is selected. In this case, in order to replace the deformed joint with an artificial knee joint, it is necessary to incise the front surface of the knee, shave the femur and/or the tibia, and the like, and thus, tools to be used in an operation for the above are required.

In the total knee replacement, the femur and/or the tibia is/are shaved in order to attach an artificial knee joint, and the extent of shaving can be determined according to, for example, the length of the posterior cruciate ligament. Since a dynamic image analysis result includes positional information on the position of an interest region, the controller 71 (outputter 712) acquires the length of the posterior cruciate ligament and outputs the amount by which the femur and/or the tibia is/are to be shaved as preoperative support information.

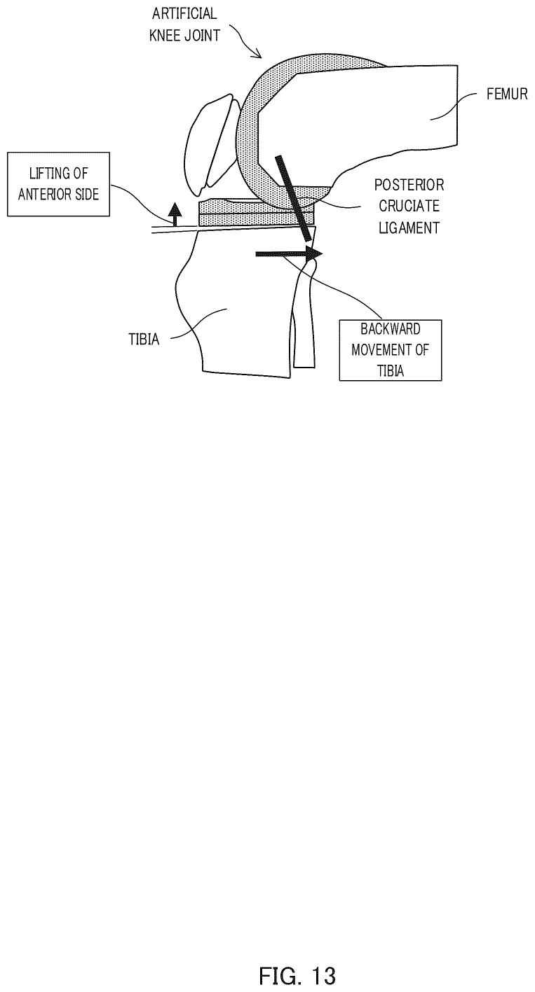

In a case where the length of the posterior cruciate ligament is short with respect to the artificial knee joint, the anterior side of the artificial knee joint is lifted, and the knee joint cannot be bent by 90° or more, but in a case where the length of the posterior cruciate ligament is appropriate, backward movement of the tibia enables deep bending of the knee joint (see FIG. 13). As described above, a dynamic image analysis result not only serves for determining the surgical procedure of an operation but also can provide a numerical value target required for an operation as preoperative support information. Further, the state of the posterior cruciate ligament before and after an operation can also be evaluated by capturing a dynamic image of the knee joint after the operation and analyzing the image.

Next, the controller 71 (outputter 712) causes the display 74 to display the above-described preoperative support information and causes the preoperative support information to be presented to the doctor.

The doctor finally determines the surgical procedure of the operation and a tool necessary for the operation by referring to the surgical procedure of the operation and the tool necessary for the operation in the preoperative support information displayed on the display 74. In addition, the doctor finally determines the operating room and the surgical staff by referring to the estimated operative time, the operating room, and the number of surgical staff in the preoperative support information displayed on the display 74, and for example, reserves the operating room or secures the surgical staff.

As described above, the preoperative support apparatus 70 includes the information acquirer 711 and the outputter 712. The information acquirer 711 acquires a dynamic image analysis result analyzed by the analyzer 312 of the radiographic image analyzing apparatus 30 and patient information included in the examination order information inputted from the radiation information terminal 60. The outputter 712 outputs preoperative support information for an operation to the display 74 based on the acquired dynamic image analysis result and patient information.

As described above, the preoperative support apparatus 70 outputs preoperative support information for an operation to the display 74 based on a dynamic image analysis result and patient information. In this way, since preoperative support information for supporting the creation of an operation plan is provided before an operation, improvement in the efficiency, safety, and reliability of the operation can be attempted in the actual operation. In addition, since it is possible to attempt improvement in the efficiency, safety, and reliability of an operation, the operation proceeds as scheduled and the operation ends as scheduled. As a result, it is possible to reduce the anxiety of a patient or the patient's family, to reduce the burden on a doctor or surgical staff by influencing the schedule of the next operation or suppressing long-time labor, and to attempt a reduction in the cost.

Any of the embodiment described above is only illustration of an exemplary embodiment for implementing the present invention, and the technical scope of the present invention shall not be construed limitedly thereby. That is, the present invention can be implemented in various forms without departing from the gist or the main features thereof.

Although embodiments of the present invention have been described and illustrated in detail, the disclosed embodiments are made for purpose of illustration and example only and not limitation. The scope of the present invention should be interpreted by terms of the appended claims.

What is claimed is:

1. A preoperative support apparatus, comprising at least one hardware processor,
wherein
the at least one hardware processor
acquires a dynamic image analysis result of a patient and patient information on the patient, and
outputs preoperative support information for an operation based on the dynamic image analysis result and the patient information which have been acquired.

2. The preoperative support apparatus according to claim 1, wherein:
the dynamic image analysis result includes change information on a change in an interest region in a dynamic image, and
the at least one hardware processor determines a state of the interest region based on the change information and the patient information and outputs the preoperative support information for the interest region.

3. The preoperative support apparatus according to claim 2, wherein
the change information is a movement amount of the interest region in the dynamic image.

4. The preoperative support apparatus according to claim 2, wherein:
the dynamic image is a dynamic image of a chest, and
the change information is an area of a region, the region being a region in which a movement amount of a lung field associated with respiration is smaller than a threshold in the dynamic image.

5. The preoperative support apparatus according to claim 2, wherein:
the dynamic image is a dynamic image of a chest, and
the change information is a change amount related to ventilation of a lung field associated with respiration or a blood flow of the lung field associated with the respiration in the dynamic image.

6. The preoperative support apparatus according to claim 2, wherein:
the dynamic image analysis result includes positional information on a position of the interest region, and
the at least one hardware processor outputs the positional information as the preoperative support information.

7. The preoperative support apparatus according to claim 1, wherein
the preoperative support information includes, in addition to information on a surgical procedure of the operation, at least one of information on a tool to be used in the surgical procedure, information on an estimated operative time, information on an operating room, and/or information on surgical staff.

8. The preoperative support apparatus according to claim 1, wherein
the patient information includes at least one of information on a chronic disease of the patient to be imaged, information on a contraindication of the patient to be imaged, information on a complication of the patient to be imaged, and/or information on medication of the patient to be imaged.

9. A preoperative support method, comprising:
acquiring a dynamic image analysis result of a patient and patient information on the patient; and
outputting preoperative support information for an operation based on the dynamic image analysis result and the patient information which have been acquired.

10. A non-transitory computer-readable recording medium storing a preoperative support program that causes a computer to execute:
acquiring a dynamic image analysis result of a patient and patient information on the patient; and
outputting preoperative support information for an operation based on the dynamic image analysis result and the patient information which have been acquired.
